(19) United States
(12) Patent Application Publication
(10) Pub. No.: US 2025/0000763 A1
(43) Pub. Date: Jan. 2, 2025

(54) TEETH WHITENING COMPOSITIONS, KITS, AND SYSTEMS

(71) Applicant: ZEST IP HOLDINGS, LLC, Carlsbad, CA (US)

(72) Inventors: Yuping LI, Irvine, CA (US); Nicholas ARALIS, San Juan Capistrano, CA (US); Wen GU, Riverside, CA (US)

(21) Appl. No.: 18/699,873

(22) PCT Filed: Oct. 21, 2022
(86) PCT No.: PCT/US2022/047427

§ 371 Date: Apr. 9, 2024

Related U.S. Application Data
(63) Continuation-in-part of application No. 17/578,303, filed on Jan. 18, 2022.
(60) Provisional application No. 63/271,038, filed on Oct. 22, 2021.
(60) Provisional application No. 63/272,470, filed on Oct. 27, 2021.

(51) Int. Cl. A61K 8/22; A61K 8/42; A61Q 11/00

(52) U.S. Cl. CPC A61K 8/22; A61K 8/42; A61Q 11/00; A61K 2800/48

(57) ABSTRACT

Dental whitening compositions, kits and systems exhibiting stability in viscosity and hydrogen peroxide concentration for an extended shelf life without refrigeration or other special storage conditions are provided herein. The composition may be in gel form and comprise a hydrogen peroxide or a hydrogen peroxide precursor, and a thickening agent comprising a crosslinked and/or water insoluble N-vinyl heterocyclic homo- or copolymer. In some aspects, a kit may be provided comprising a two-part dental whitening system, wherein one part comprises a whitening agent, and a separate part comprises a strong base.

### CROSS-REFERENCE TO RELATED APPLICATION

This application claims priority to U.S. patent application Ser. No. 17/578,303, filed on Jan. 18, 2022, which claims priority to U.S. provisional application Nos. 63/271,038, filed on Oct. 22, 2021 and 63/272,470, filed on Oct. 27, 2021. These and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### FIELD OF THE INVENTION

The field of the invention is teeth whitening compositions, kits, and systems.

### BACKGROUND

Teeth can become discolored as a consequence of food consumption, tobacco use, hemorrhages or disorders during odontogenesis, or intake of certain medications. Teeth whitening is often desirable for aesthetic purposes, and in recent decades, it has become increasingly popular, whether through dental office visits or certain at-home kits. Teeth whitening usually involves an application of a bleaching active ingredient to the surface of the teeth or from the inside of teeth (e.g., endodontic bleaching) to achieve a desired whitening effect.

The most commonly used bleaching ingredients are peroxide, such as hydrogen peroxide, carbamide peroxide, sodium perborate and sodium percarbonate. They can bleach the teeth, remove stains and kill bacteria. However, peroxide is highly reactive and therefore may be difficult to formulate.

Some have noted that hydrogen peroxide can spontaneously decompose into hydrogen cation (H⁺) and perhydroxyl anion (HO₂⁻), which further decomposes to free radical of hydroxyl (HO)· and perhydroxyl (HO₂)· when it reacts with another hydrogen peroxide. Both free radicals are unstable oxidants, which can break down the carbon-carbon double bonds of most stains, reduce their light absorption and increase tooth whiteness.

Teeth whitening products can range from over-the-counter (OTC) toothpastes, mouthwashes, whitening strips, whitening pens and whitening syringes, to dentist-prescribed at-home tooth whitening trays, or in-office whitening products with high concentration of hydrogen peroxide. For safety and effectiveness purposes, it can be beneficial for whitening agents to be formulated into gel or paste forms so they can adhere or remain in contact with tooth surfaces without running to adjacent soft tissue in the oral cavity.

A whitening gel with a pH below 5.5 tends to demineralize tooth enamel, increase tooth surface roughness, reduce its hardness, and reduce fracture resistance. Such gels can also lead to tooth sensitivity and discomfort for the patient or other user. Furthermore, a low pH of a bleaching gel can reduce its bleaching efficacy by inhibiting the formation of hydroxyl radicals and perhydroxyl radicals, the primary species responsible for the bleaching outcomes. Unfortunately, because hydrogen peroxide has poor stability, commercially available hydrogen peroxide containing dental whitening products typically have a low pH in order to maintain adequate shelf life.

Carbamide peroxide, a hydrogen peroxide precursor, is stable when stored in a refrigerator. However, it breaks down in the presence of water and releases hydrogen peroxide. Therefore, most carbamide peroxide containing whitening products have to be formulated in anhydrous compositions to achieve an acceptable shelf life. Due to its low water content and low peroxide content, it requires greater than 60 minutes of bleaching treatment time which can lead to tooth sensitivity, tooth dehydration, and the like.

Vinylpyrrolidone polymer, as called polyvinylpyrrolidone (PVP), has been used as a thickener in certain tooth whitening compositions in its linear form. U.S. Pat. No. 6,500,408 to Chen describes a tooth bleach composition that utilizes linear PVP as a thickening agent, with the linear PVP present in the composition at a concentration of 25% or greater. The linear PVP thickened whitening formulation disadvantageously forms sticky or tacky gel, limiting its ability to spread around the teeth for bleach and causing gel stringing and roping when dispensed from syringes to the dental whitening tray.

U.S. Pat. No. 9,616,005 describes using a solid particulate rheology-modifying agent (fumed silica and/or fumed alumina) in teeth whitening compositions thickened by linear PVP polymers in an effort to reduce the stringiness and improve long-term rheological stability. Such compositions are known to be more difficult and expensive to formulate as they require the addition of inorganic thickeners.

In an effort to address aging stability of peroxide in tooth whitening compositions, polyvinylpyrrolidone hydrogen peroxide complexes (PVP-H₂O₂) have been developed and used as an active ingredient in tooth whitening compositions. The PVP-H₂O₂ complexes, also known as peroxydone complexes, are made by reacting the PVP powder with an aqueous H₂O₂ solution and then drying out to form free-flowing powders. In anhydrous conditions, the PVP-H₂O₂ complexes are stable as hydrogen peroxide is stabilized with PVP polymer via hydrogen bonds. The PVP-H₂O₂ complexes have been used widely in tooth whitening compositions, including compositions described in U.S. Pat. Nos. 10,758,472 and 8,137,658, and U.S. Pat. Publication Nos. 2018/0168993, 2006/0062744, and 2017/0172864. However, the PVP-H₂O₂ complexes are unstable in aqueous solutions. Therefore, such whitening compositions are known to be formulated in a substantially non-aqueous condition where the moisture range of the whitening compositions is about 6% or less. Such anhydrous whitening compositions may adversely dehydrate the treated tooth and increase the likelihood of tooth sensitivity.

U.S. Pat. No. 8,574,555 to Longo et al describes an aqueous tooth whitening composition containing a hydrogen peroxide solution, and multiple thickening agents, including polyacrylic acid and powdered PVP-H₂O₂ complexes. Longo's compositions comprise a combination of bleaching agent stabilizers in a precise ratio to the available hydrogen peroxide. Because the PVP-H₂O₂ complex prevents ionization of un-neutralized polyacrylic acid backbone, pre-neutralization of polyacrylic acid before combining with the PVP-H₂O₂ complex is needed which makes manufacturing complicated.

U.S. Pat. Publication No. 2013/0209376 to Prosise et al describes specific oral care compositions comprising a strongly swellable, lightly to moderately crosslinked polyvinylpyrrolidone and an oral care ingredient, e.g. abrasives (also known as polishing agents). However, abrasives commonly used in oral care compositions are known to facilitate the destabilization of peroxide compounds.

U.S. Pat. Publication No. 2004/0062723 describes a dual component whitening dentifrice composition comprised of a first dentifrice component containing a peroxide compound and a second dentifrice component containing an abrasive, such as alumina or silica, which is incompatible with the peroxide. The first and second dentifrice components are to be maintained separately until dispensed and combined for application to teeth. Such dual component whitening compositions comprise low concentrations of peroxide compound (e.g., about 5-10% by weight of the dentifrice component).

The main challenge in tooth whitening systems with high concentrations of peroxide (e.g., 20-40%) is often considered to be to obtain stable gels. Where it is desired to produce highly concentrated yet stable hydrogen peroxide bleaching compositions, it was known to be necessary to replace polymeric thickeners with non-polymeric ones such as fumed silica in order to obtain stable gels. Polymeric thickeners are susceptible to degradation over time and are therefore unable to maintain its gelling capabilities in the presence of concentrated acidic aqueous hydrogen peroxide solutions.

### SUMMARY

Accordingly, teeth whitening compositions, kits and systems with improved stability, manufacturability, consistency, spreadability, whitening effectiveness and/or handleability are disclosed herein. Also disclosed herein are teeth whitening compositions with neutral or near-neutral pH that can be stored at room temperature over an extended period of time while maintaining a large percentage of its available hydrogen peroxide content and/or viscosity. In some embodiments, the composition is a gel. In some embodiments, the composition is a paste.

In one aspect, a ready to use, one part, aqueous tooth whitening composition is provided, comprising at least one whitening agent, at least one thickening agent, and a liquid carrier. In some embodiments, the ready to use tooth whitening composition is formulated to maintain at least 90% available hydrogen peroxide content and at least 80% viscosity after 24 months storage at 23° C. In some embodiments, the ready to use tooth whitening composition is formulated to maintain at least 90% available hydrogen peroxide content and at least 80% viscosity after 4 weeks storage at 60° C. In some embodiments, the ready to use tooth whitening composition is formulated to maintain at least 90% available hydrogen peroxide content and at least 80% viscosity after 36 months storage at 23° C. In some embodiments, the ready to use tooth whitening composition is formulated to maintain greater than 90% available hydrogen peroxide content after 4 weeks storage at 60° C. with no significant loss of gel viscosity. In some embodiments, the compositions described herein can be manufactured at a relatively low cost and simplified process.

In some embodiments, the at least one whitening agent is present in an amount between 3-50 wt % of the composition. In some embodiments, the at least one whitening agent is present in an amount between 6-40 wt % of the composition. In some embodiments, the at least one whitening agent is present in an amount between 6-25 wt % of the composition. In some embodiments, the at least one whitening agent is present in an amount between 5-25 wt % of the composition. In some embodiments. the at least one whitening agent is present in an amount between 6-15 wt % of the composition. In some embodiments, the at least one whitening agent is present in an amount between 9-15 wt % of the composition. In some embodiments, the at least one whitening agent is present in an amount of at least 6 wt % of the composition. In some embodiments, the at least one whitening agent is present in an amount of at least 9 wt % of the composition. In some embodiments, the at least one whitening agent comprises hydrogen peroxide. In some embodiments, the at least one whitening agent is hydrogen peroxide. In some embodiments, the at least one whitening agent composes a hydrogen peroxide aqueous solution. In some embodiments, the at least one whitening agent comprises at least one of hydrogen peroxide and urea hydrogen peroxide.

In some embodiments, the at least one whitening agent comprises a peroxide compound (e.g., hydrogen peroxide), and the peroxide compound is present in an amount between 3-50 wt % of the composition. In some embodiments, the peroxide compound is present in an amount between 6-40 wt % of the composition. In some embodiments, the peroxide compound is present in an amount between 6-25 wt % of the composition. In some embodiments, the peroxide compound is present in an amount between 5-25 wt % of the composition. In some embodiments, the peroxide compound is present in an amount between 6-15 wt % of the composition. In some embodiments, the peroxide compound is present in an amount between 9-15 wt % of the composition. In some embodiments, the peroxide compound is present in an amount of at least 6 wt % of the composition. In some embodiments, the peroxide compound is present in an amount of at least 9 wt % of the composition.

In some embodiments, the at least one thickening agent is present in an amount between 0.1-15 wt % of the composition. In some embodiments, the at least one thickening agent is present in an amount between 0.1-10 wt % of the composition. In some embodiments, the at least one thickening agent comprises a N-vinyl heterocyclic homo- or copolymer. In some embodiments, the at least one thickening agent is water insoluble. In some embodiments, the at least one thickening agent comprises a crosslinked vinylpyrrolidone polymer and/or vinylpyrrolidone containing copolymer. In some embodiments, the at least one thickening agent comprises a chemically crosslinked vinylpyrrolidone polymer and/or vinylpyrrolidone containing copolymer. In some embodiments, the at least one thickening agent comprises a physically crosslinked vinylpyrrolidone polymer and/or vinylpyrrolidone containing copolymer. Without limiting the composition, mechanism, or function of the invention, it is believed that the crosslinking renders the thickener water insoluble, which helps to prevent degradation of thickener in the presence of concentrated peroxide agents. In some embodiments, the at least one thickening agent comprises a non-crosslinked, water insoluble vinylpyrrolidone containing copolymer. It is generally understood that crosslinked polymers are insoluble in water. However, a non-crosslinked vinylpyrrolidone containing copolymer may be water insoluble when it contains large amount of hydrophobic domains.

In some embodiments, the at least one thickening agent does not include anhydrous powder of PVP-H₂O₂ complexes. In some embodiments, the composition does not include anhydrous powder of PVP-H₂O₂ complexes. In some embodiments, the at least one thickening agent does not include linear and/or water soluble PVPs.

In some embodiments, the tooth whitening composition lacks a non-polymeric thickener. In some embodiments, the only thickeners present in a composition are N-vinyl heterocyclic homo- or copolymer. In some embodiments, the only thickeners present in a composition are water insoluble N-vinyl heterocyclic homo- or copolymer. In some embodiments, the only thickeners present in a composition are crosslinked N-vinyl heterocyclic homo- or copolymers. As used herein, the term “crosslinked N-vinyl heterocyclic homo- or copolymers” refers to N-vinyl heterocyclic homo- or copolymers with any suitable degree of crosslinking (e.g., lightly, moderately, substantially, lightly to moderately, moderately to substantially, lightly to substantially). As used herein, the term “crosslinked polyvinylpyrrolidone” refers to polyvinylpyrrolidone with any suitable degree of crosslinking. The crosslinking can be physical crosslinking and/or chemical crosslinking.

The liquid carrier can comprise one or more of water, glycerin, propylene glycol, polyethylene glycol, sorbitol, ethanol and/or other suitable liquid carriers. In some embodiments, the liquid carrier is present in an amount of between 1.0-95 wt % of the composition. In some embodiments, the liquid carrier is present in an amount of between 30-95 wt % of the composition. In some embodiments, the liquid carrier is present in an amount of between 1.0-85 wt % of the composition. In some embodiments, the liquid carrier is present in an amount of at least 10 wt % of the composition. In some embodiments, the liquid carrier is present in an amount of at least 20 wt % of the composition. In some embodiments, the liquid carrier is present in an amount of at least 30 wt % of the composition. In some embodiments, the liquid carrier is present in an amount of at least 40 wt % of the composition. In some embodiments, the liquid carrier is present in an amount of at least 50 wt % of the composition. In some embodiments, the liquid carrier comprises water. In some embodiments, the liquid carrier comprises at least one hygroscopic agent selected from the polyol group consisting of glycerin, propylene glycol, polyethylene glycol, polypropylene glycol and sorbitol. In some embodiments, water is present in an amount of at least 10 wt % of the composition. In some embodiments, water is present in an amount of at least 20 wt % of the composition. In some embodiments, water is present in an amount of at least 30 wt % of the composition. In some embodiments, water is present in an amount of at least 40 wt % of the composition. In some embodiments, water is present in an amount of at least 50 wt % of the composition.

In some embodiments, the composition has a pH of at least 6.0. In some embodiments, the composition has a pH of at least 6.2. In some embodiments, the composition has a pH of at least 6.5. In some embodiments, the composition includes a pH adjusting agent selected from the group consisting of triethanolamine, sodium hydroxide, potassium hydroxide, and ammonium hydroxide. In some embodiments, a pH adjusting agent is present in an amount of between 0.01 and 5 wt % of the composition.

In another aspect of the disclosure, a tooth whitening kit is provided comprising a first part and a second part. The first part comprises at least one whitening agent, and the second part comprises an alkaline material. At least one of the first and second parts comprises an effective amount of at least one thickening agent.

In some embodiments, the at least one thickening agent comprises N-vinyl heterocyclic homo- or copolymer. In some embodiments, each of the first and second parts comprises an amount of at least one thickening agent. In some embodiments, the thickening agent is present in the first part in an amount between 5 wt % to 15 wt %, or between 5 wt % to 10 wt %. In some embodiments, the thickening agent is present in the second part in an amount between 1 wt % to 15 wt %, or between 2 wt % to 10 wt %. In some embodiments, the at least one thickening agent is present in an amount between 0.1-15 wt % of the activated composition. In some embodiments, the at least one thickening agent is present in an amount between 0.1-10 wt % of the activated composition. In some embodiments, the at least one thickening agent is water insoluble. In some embodiments, the at least one thickening agent is crosslinked. In some embodiments, the at least one thickening agent comprises a crosslinked vinylpyrrolidone polymer and/or vinylpyrrolidone containing copolymer. In some embodiments, the at least one thickening agent does not include anhydrous powder of PVP-H₂O₂ complexes. In some embodiments, the activated composition does not include anhydrous powder of PVP-H₂O₂ complexes. In some embodiments, the first part does not include anhydrous powder of PVP-H₂O₂ complexes. In some embodiments, the second part does not include anhydrous powder of PVP-H₂O₂ complexes. In some embodiments, the first part does not include linear (water soluble) PVP. In some embodiments, the second part does not include linear PVP. In some embodiments, the activated composition does not include linear PVP.

In some embodiments, the tooth whitening composition lacks a non-polymeric thickener. In some embodiments, the only thickeners present in a composition are N-vinyl heterocyclic homo- or copolymer. In some embodiments, the only thickeners present in a composition are water insoluble N-vinyl heterocyclic homo- or copolymer. In some embodiments, the only thickeners present in a composition are crosslinked N-vinyl heterocyclic homo- or copolymer.

In some embodiments, the activated composition has a pH of at least 6.0. In some embodiments, the composition has a pH of at least 7. In some embodiments, the activated composition has a pH of at least 8. In some embodiments, the activated composition has a pH of between about 7 to about 9. In some embodiments, at least one of the first part and the second part includes a pH adjusting agent selected from the group consisting of triethanolamine, sodium hydroxide (NaOH), potassium hydroxide (KOH), and ammonium hydroxide. In some embodiments, a pH adjusting agent is present in an amount of between 0.01 and 5 wt % of the activated composition.

In some embodiments, the first part and second part are stored separately, and combining the first part and the second part forms an activated tooth whitening composition with a pH of between 7 and 9. In some embodiments, the activated tooth whitening composition is formed by combining the first part and the second part in a volume ratio of between 3.5:1 and 4.5:1. In some embodiments, the kit comprises a dual barrel syringe that stores the first part and second part separately. In some embodiments, the dual barrel syringe comprises a mixing tip. In some embodiments, a pH of the activated tooth whitening composition increases during the first twenty minutes after the activated tooth whitening composition is formed. In some embodiments, the first part is a gel. In some embodiments, the second part is a gel. In some embodiments, the first part and second part are formulated such that when stored separately for at least 24 months at 23° C., then combined, the activated (combined and/or mixed) composition comprises at least 90% available hydrogen peroxide content and at least 80% viscosity relative to the available hydrogen peroxide and viscosity if combined prior to storage for the at least 24 months at 23° C. In some embodiments, the first part and second part are formulated such that when stored separately for at least 8 weeks at 50° C., then combined, the activated (combined and/or mixed) composition comprises at least 90% available hydrogen peroxide content and at least 80% viscosity relative to the available hydrogen peroxide and viscosity if combined prior to storage for the at least 8 weeks storage at 50° C. In some embodiments, the first part and second part are formulated such that when stored separately for at least 36 months storage at 23° C., then combined, the activated (combined and/or mixed) composition comprises at least 90% available hydrogen peroxide content and at least 80% viscosity relative to the available hydrogen peroxide and viscosity if combined prior to storage for the at least 36 months storage at 23° C. In some embodiments, the first part and second part are formulated such that when stored separately for at least 8 weeks at 50° C., then combined, the activated (combined and/or mixed) composition comprises at least 90% available hydrogen peroxide content with no significant loss of gel viscosity relative to the available hydrogen peroxide and viscosity if combined prior to storage for the at least 8 weeks at 50° C.

Viewed from another perspective, the first and second part can be formulated to be highly stable when separate, yet highly reactive when combined for effective tooth whitening. In some aspects, the activated composition should be used immediately or shortly upon activation (combining and/or mixing). In some aspects, the activated compositions need only be stable for about 20 minutes, or about 30 minutes upon activation (sufficient time to whiten teeth).

In some embodiments, the whitening agent is and/or comprises a peroxide. In some embodiments, the first part can have an acidic pH and include at least about 25 wt % available hydrogen peroxide. In some embodiments, the second part comprises a strong base dispersed in a stable gel, wherein the strong base comprises at least one of an alkali metal oxide, an alkali metal hydroxide, an alkaline earth metal oxide, and an alkaline earth metal hydroxide. In some embodiments, the strong base is selected from the group consisting of triethanolamine, sodium hydroxide, potassium hydroxide, and ammonium hydroxide.

In some embodiments, the whitening agent is and/or comprises a peroxide, and the concentration of peroxide compound present in the first part ranges from 25 wt % to 50 wt %. In some embodiments, the whitening agent is a peroxide, and the concentration of peroxide compound present in the first part ranges from 25 wt % to 40 wt %. In some embodiments, the peroxide compound is hydrogen peroxide and the concentration of hydrogen peroxide present in the whitening component ranges from 25 wt % to 40 wt %. In some embodiments, the peroxide compound is hydrogen peroxide and the concentration of hydrogen peroxide present in the whitening component is at least 20 wt % of the whitening component. In some embodiments, the peroxide compound is hydrogen peroxide and the concentration of hydrogen peroxide present in the whitening component is at least 25 wt % of the whitening component. In some embodiments, the at least one whitening agent comprises hydrogen peroxide. In some embodiments, the at least one whitening agent is hydrogen peroxide. In some embodiments, the at least one whitening agent comprises at least one of hydrogen peroxide and urea hydrogen peroxide. In some embodiments, the at least one whitening agent composes a hydrogen peroxide aqueous solution.

In some embodiments, the at least one whitening agent is present in an amount ranging from 25-50 wt % of the activated composition. In some embodiments, the at least one whitening agent is present in an amount ranging from 25-40 wt % of the activated composition. In some embodiments, the at least one whitening agent is present in an amount between 30-40 wt % of the activated composition. In some embodiments, the at least one whitening agent is present in an amount between 35-40 wt % of the activated composition. In some embodiments, the at least one whitening agent is present in an amount of at least 25 wt % of the activated composition. In some embodiments, the at least one whitening agent is present in an amount of at least 30 wt % of the activated composition. In some embodiments, the at least one whitening agent comprises a peroxide compound, and the peroxide compound is present in an amount between 25-50 wt % of the activated composition. In some embodiments, the peroxide compound is present in an amount between 25-40 wt % of the activated composition. In some embodiments, the peroxide compound is present in an amount between 30-40 wt % of the activated composition. In some embodiments, the peroxide compound is present in an amount between 35-40 wt % of the activated composition. In some embodiments, the peroxide compound is present in an amount of at least 25 wt % of the activated composition. In some embodiments, the peroxide compound is present in an amount of at least 35 wt % of the activated composition.

In some embodiments, the second part comprises one or more liquid carriers, and wherein the one or more liquid carriers are present in an amount of between 50 wt % and 90 wt % of the second part. In some embodiments, the second part has a pH of at least 12. The liquid carrier can comprise one or more of water, glycerin, propylene glycol, polyethylene glycol, sorbitol, ethanol and/or other suitable liquid carriers. In some embodiments, the liquid carrier is present in an amount of between 1.0-99 wt % of the second part. In some embodiments, the liquid carrier is present in an amount of between 1.0-95 wt % of the second part. In some embodiments, the liquid carrier is present in an amount of between 30-95 wt % of the second part. In some embodiments, the liquid carrier is present in an amount of between 40-90 wt % of the second part. In some embodiments, the liquid carrier is present in an amount of between 1.0-85 wt % of the second part. In some embodiments, the liquid carrier is present in an amount of at least 10 wt % of the second part. In some embodiments, the liquid carrier is present in an amount of at least 20 wt % of the second part. In some embodiments, the liquid carrier is present in an amount of at least 30 wt % of the second part. In some embodiments, the liquid carrier is present in an amount of at least 40 wt % of the second part. In some embodiments, the liquid carrier is present in an amount of at least 50 wt % of the second part. In some embodiments, the liquid carrier comprises water. In some embodiments, the liquid carrier comprises at least one hygroscopic agent selected from the polyol group consisting of glycerin, propylene glycol, polyethylene glycol, polypropylene glycol and sorbitol. In some embodiments, water is present in an amount of at least 10 wt % of the second part. In some embodiments, water is present in an amount of at least 20 wt % of the second part. In some embodiments, water is present in an amount of at least 30 wt % of the second part. In some embodiments, water is present in an amount of at least 40 wt % of the second part. In some embodiments, water is present in an amount of at least 50 wt % of the second part.

In some embodiments, the liquid carrier is present in the activated composition in an amount of between 50-95 wt %. In some embodiments, the liquid carrier is present in the activated composition in an amount of between 50-90 wt %. In some embodiments, the liquid carrier is present in the activated composition in an amount of between 40-90 wt %. In some embodiments, the liquid carrier is present in the activated composition in an amount of at least 10 wt %. In some embodiments, the liquid carrier is present in the activated composition in an amount of at least 20 wt %. In some embodiments, the liquid carrier is present in the activated composition in an amount of at least 30 wt %. In some embodiments, the liquid carrier is present in the activated composition in an amount of at least 40 wt %. In some embodiments, the liquid carrier is present in the activated composition in an amount of at least 50 wt %. In some embodiments, the liquid carrier is present in the activated composition in an amount of between 40-60 wt %. In some embodiments, the liquid carrier is present in the activated composition in an amount of between 40-50 wt %. In some embodiments, the liquid carrier is present in the activated composition in an amount of about 50 wt %.

In some embodiments, the kit is an at-home. In some embodiments, the kit is an in-office kit. In some embodiments, an apparatus for applying the composition, and instructions for determining a composition application time to achieve a whitening effect may be provided.

Instead of using the anhydrous powder of PVP-H₂O₂ complexes which are costly and requires additional manufacturing process, including synthesis, purification, drying etc., or using soluble/linear PVP polymers which form highly stringy dental bleaching compositions, some preferred compositions and kits described herein include a readily available, crosslinked and/or water insoluble N-vinyl heterocyclic homo- or copolymer that is compatible with hydrogen peroxide and forms stable non-stringy gels with them as a thickening agent. Water insoluble N-vinylpyrrolidone homo- or copolymers with variable swellability may be prepared by copolymerizing N-vinylpyrrolidone monomers in aqueous or organic solvents with or without difunctional vinyl or acrylic compounds acting as crosslinking agents.

It has unexpectedly been found that using a crosslinked and/or water insoluble N-vinyl pyrrolidone polymer as a rheological thickener yields much more stable tooth whitening compositions in aqueous condition. The known polymeric gel-forming agents, such as carboxypolymethylene or soluble/linear PVP, are susceptible to be degraded in the presence of highly concentrated peroxide agents. Although the dental whitening compositions thickened by these polymers have acceptable initial viscosity, they quickly become runny, which greatly reduces user acceptance and shelf life. In contrast, tooth whitening compositions thickened by a crosslinked and/or water insoluble N-vinyl pyrrolidone homo- or copolymers have been found to impart surprisingly great aging stability in viscosity and hydrogen peroxide concentration, therefore increasing their shelf-life and whitening efficacy.

Although bleaching stabilizers, such as ethylenediaminetetraacetic acid (EDTA), may be included in the compositions and kits disclosed herein, it has been found that some compositions and kits of the disclosure do not require a bleaching stabilizer to be present in the composition in a precise ratio range to the available hydrogen peroxide in order to achieve long term stability. In some embodiments, where a bleaching stabilizer (e.g., ETDA, disodium EDTA) is present, a ratio of the peroxide compound to the stabilizer(s) is at least 65:1, or at least 70:1.

In some aspects, tooth whitening compositions and kits disclosed herein exhibit high temperature stability. In one preferred composition, a one-part whitening gel with a hydrogen peroxide concentration of about 9 to about 14 wt % maintained at least 90% available hydrogen peroxide content, and at least 80% viscosity after 4 weeks of storage at 60° C. Based on an accelerated stability testing protocol (ASTM F1980-6), it assumes that the chemical reaction in the aging process of a material follow the Arrhenius reaction rate function. For accelerated aging of materials, this can be expressed in equation (1).

$${Q}_{10} = {( \frac{{t}_{AA}}{{t}_{RT}} )}^{\frac{10}{{T}_{AA} - {T}_{RT}}} \qquad ( 1 )$$

Where Q₁₀=Temperature Coefficient for such chemical reactions;
T_{AA}=Temperature of Accelerated Aging;
T_{RT}=Temperature of Recommended Storage, usually room temperature;
t_{AA}=the accelerated aging time; and
t_{RT}=the real aging time.

In some aspects, whitening composition disclosed herein can survive 14 weeks of storage at 47° C., or 29 weeks at 40° C., or a minimum of three years storage at room temperature without refrigeration. In some aspects, a whitening gel composition in accordance with the disclosure maintained greater than 90% available hydrogen peroxide content after 4 weeks storage at 60° C. with no significant loss of gel viscosity (i.e., no greater than 10% loss of gel viscosity). In some aspects, the composition can be formulated as a paste.

Other advantages and benefits of the disclosed compositions, kits, and systems will be apparent to one of ordinary skill with a review of the following detailed description.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing of which:

FIG. 1 shows the pH of the mixed composition of Example 7 over time.

### DETAILED DESCRIPTION

After reading this description, it will become apparent to one skilled in the art how to implement the disclosed compositions, kits, and systems in various alternative embodiments and alternative applications. However, all the various embodiments of the present disclosure will not be described herein. It is understood that the embodiments presented here are presented by way of an example only, and not limitation. As such, this detailed description of various alternative embodiments should not be construed to limit the scope or breadth of the present disclosure as set forth below.

In some embodiments, a stable one-part aqueous dental bleaching composition having a pH of between 6-7, at least 6, at least 6.2, at least 6.5, or at least 7 is provided. The stability of several contemplated compositions has been proven under accelerated aging conditions that translate into at least a three years shelf life for the composition under typical room temperature storage conditions. Based on the accelerated stability testing protocol described herein, for a tooth whitening product that survives (retains at least 90% hydrogen peroxide content and at least 80% viscosity) for a minimum of 4 weeks at 60° C., it is assumed that it would survive on the open market for a minimum of three years under room temperature storage conditions.

In some aspects, contemplated stable tooth whitening compositions can comprise a bleaching agent, a polymeric thickening agent, and a liquid carrier. In some embodiments, the pH of the tooth bleaching composition during the bleaching process can be in a range of pH 6.0-9.0. In some embodiments, the pH of the tooth bleaching composition during the entire bleaching process can be in a range of pH 6.0-9.0.

In some aspects, a one-part tooth whitening system is provided comprising a bleaching agent, a polymeric thickening agent, a liquid carrier, and a pH adjusting agent. The one-part bleaching system can have a pH above 6.0 and can be storage stable in hydrogen peroxide concentration and viscosity.

In accordance with another aspect, the disclosure relates to a polymeric thickener, a water insoluble N-vinyl heterocyclic homo- or copolymer derived from a N-heterocyclic vinyl monomer, preferably comprising N-vinyl heterocyclic monomers having from 3 to 7 atoms in a heterocyclic ring, including a carbonyl carbon atom and a nitrogen heteroatom containing a vinyl group. Suitable water insoluble N-vinyl heterocyclic polymers include, but are not limited to, crosslinked (e.g., lightly, lightly to moderately, moderately, highly) poly-N-vinyl pyrrolidone, poly-N-2-caprolactam and mixture thereof. In a preferred embodiment, the polymer is the crosslinked polyvinylpyrrolidone or “cPVP”, preferably a crosslinked N-vinyl pyrrolidone homo- or copolymer.

Without wishing to be bound by any particular theory, composition or function, it is believed that the chemical and amphiphilic characteristics of the crosslinked and/or water insoluble vinylpyrrolidone polymers play a critical role in long term stability of the whitening compositions. The crosslinked and/or water insoluble vinylpyrrolidone polymer has hydrophilic lactam groups of the pyrrolidone ring that bind to peroxide for maintaining the peroxide available to whiten the teeth. Their hydrophobic groups from methylene groups of the pyrrolidone ring, the aliphatic backbone, crosslinkers and/or copolymers inhibit these thickeners from hydrolytic degradation with peroxide solution. Hydrolysis is defined as the reaction with water where the water molecules lead to cleavage of chemical bonds within a polymer. Catalysts, such as peroxide or any ions, can accelerate polymer degradation, causing the loss of viscosity. The hydrolysis rate depends on both the susceptible chemical bonds within the polymer and the concentration of water inside the polymer matrix. Soluble polymeric thickeners are fully dissolved in water and commonly susceptible to hydrolytic degradation in the presence of highly concentrated peroxide. It is believed the hydrophobic domains of the crosslinked and/or water insoluble N-vinyl pyrrolidone polymers shielded the chemically susceptible bond from water and therefore significantly improved their stability.

In another aspect, the invention provides a stable tooth bleaching composition with improved performance. The improved performance includes, among other things, improved stability, handling, consistency, spreadability and application control. The crosslinked and/or water insoluble N-vinyl pyrrolidone polymers with the amphiphilic characteristic impart the tooth whitening compositions of the invention with great Bingham plastic properties, i.e. behaving as a rigid body at low stress and a viscous fluid at high stress. This characteristic results in the whitening composition remaining where placed without runny to undesired area. This feature is greatly benefit for whitening application which requires precise application control.

### ONE-PART TEETH WHITENING SYSTEMS

The present disclosure provides a ready to use, one-part tooth bleaching composition that is stable at typical room temperature storage conditions for an extended period of time. The “ready to use” composition can be packaged such that a user can simply dispense the composition and apply it to the user's teeth (e.g., without reconstitution, dilution, or mixing), for example, via an applicator or a brush, and/or apply the composition directly to the user's teeth from the packaging (e.g., a whitening pen). The compositions can comprise a hydrogen peroxide whitening agent that releases peroxide to the tooth surface for a desired whitening effect without appreciable decomposition when stored in plastic syringes, pens, or other suitable containers.

In some aspects, a one-part tooth whitening composition is provided, comprising at least one whitening/bleaching agent, at least one thickening agent, and a liquid carrier. A basic agent, a desensitizing agent, a flavorant, a combination thereof, and/or any other suitable ingredients may also be included.

The ready to use composition can be formulated to maintain at least 90% available hydrogen peroxide content, and at least 80% viscosity after 4 weeks of storage at 60° C., 14 weeks of storage at 47° C., or 29 weeks at 40° C. The ready to use composition can be formulated to maintain at least 90% available hydrogen peroxide content, and at least 80% viscosity after 12 months, after 24 months, or after at least 36 months of storage at 23° C., or at room temperature. The ready to use composition can be formulated as a gel with the composition maintaining greater than 90% available hydrogen peroxide content after 4 weeks storage at 60° C. with no significant loss of gel viscosity.

### Whitening or Bleaching Agent

Suitable whitening or bleaching agents for compositions, kits and systems of the disclosure include any material that whitens a tooth to which it is applied, for example, a peroxide compound. A peroxide is an oxidizing compound with the structure R—O—O—R, where R=any element. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxy phthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof.

In some contemplated embodiments, hydrogen peroxide or urea hydrogen peroxide is preferred as a whitening agent, and can be present in one-part whitening systems in an amount from about 3-50 wt %, about 5-25 wt %, about 6-15 wt %, or about 9-15 wt % of the composition. In some aspects, the hydrogen peroxide composes a 30-50 wt % hydrogen peroxide solution and the hydrogen peroxide solution comprises between 10-50 wt % of the composition, with the hydrogen peroxide comprising between 3-25 wt % of the composition.

### Thickening Agent

Compositions, kits and systems of the disclosure can include a thickening agent to yield a composition with a desired viscosity. All orally acceptable thickening agents are contemplated. In some preferred aspects, the one or more thickening agents comprise at least one crosslinked and/or water insoluble vinylpyrrolidone homo- or copolymer, preferably a crosslinked (e.g., lightly to moderately crosslinked, highly crosslinked) vinylpyrrolidone homo- or copolymer, in an amount ranging from 0.1-15 wt %, or about 0.1-10 wt % of the composition.

In some aspects, the at least one thickening agent can comprise a crosslinked polyvinylpyrrolidone, for example, a lightly to moderately crosslinked vinylpyrrolidone polymer, and the at least one thickening agent can be present in an amount ranging from 0.1-15 wt %, or about 0.1-10 wt %. Applicant surprisingly discovered that a low concentration of crosslinked vinylpyrrolidone polymer could be used, while having a clear and homogeneous gel formulation that is stable in viscosity and hydrogen peroxide concentration over long term storage at room temperature. In some aspects, the at least one thickening agent comprises a strongly swellable, lightly to moderately crosslinked vinylpyrrolidone polymer. Examples include, but are not limited to, FlexiThix™ polymer (INCI name polyvinylpyrrolidone, Ashland Specialty, Wayne, NJ), ACP-1120, ACP-1179, and ACP-1180, available from Ashland INC (Wayne, NJ).

In some aspects, the at least one thickening agent comprises a crosslinked (e.g., lightly to moderately crosslinked and/or highly crosslinked) vinylpyrrolidone polymer in a range of about 0.1-20% by weight of the one-part tooth whitening composition. In some aspects, the at least one thickening agent comprises a crosslinked (e.g., lightly to moderately crosslinked and/or highly crosslinked) vinylpyrrolidone polymer in a range of about 2-12% by weight of the one-part tooth whitening composition. In some aspects, the at least one thickening agent comprises a crosslinked (e.g., lightly to moderately crosslinked and/or highly crosslinked) vinylpyrrolidone polymer in a range of about 4-10% by weight of the one-part tooth whitening composition. In some aspects, the at least one thickening agent comprises a crosslinked (e.g., lightly to moderately crosslinked and/or highly crosslinked) vinylpyrrolidone polymer in a range of about 4-9% by weight of the one-part tooth whitening composition. It should be appreciated that the concentration of the crosslinked (e.g., lightly to moderately crosslinked and/or highly crosslinked) vinylpyrrolidone polymer may be adjusted to achieve an appropriate viscosity, stickiness, or other physical properties.

A lightly to moderately crosslinked vinylpyrrolidone polymer is a nonionic polymer and is advantageously easily dispersed in aqueous, glycols, alcohol, and glycerin-based systems. It does not require neutralization and is highly salt tolerant. Unlike linear/water soluble vinylpyrrolidone homopolymer which produces a sticky or tacky bleaching gel, the compositions of the present invention can be made less tacky with crosslinked and/or water insoluble vinylpyrrolidone polymer. It is particularly beneficial if the composition is directly applied from a pen.

The lightly to moderately crosslinked vinylpyrrolidone polymer can comprise strongly swellable, moderately crosslinked vinylpyrrolidone polymer powders characterized by an aqueous gel volume of about 15-150 ml/g of polymer and a Brookfield viscosity in 5% aqueous solution of at least 10,000 cps, as described in U.S. Pat. Nos. 5,073,614 and 5,139,770. It should be appreciated that gel volume is a measure of the swelling property of the crosslinked polymer, which can be determined, for example, by adding 1 g of the polymer to a suitable container filled with water, shaking the mixture and allowing it to stand at room temperature for 3 days. The volume of the gel produced in water is measured and taken as the gel volume.

In some aspects, the crosslinked vinylpyrrolidone polymer can comprise strongly swellable, moderately crosslinked vinylpyrrolidone polymer powders characterized by an aqueous gel volume of about 25-75, or 30-60 ml/g of polymer and a Brookfield viscosity in 5% aqueous solution of at least 15,000 cps, at least 20,000 cps, between 20,000-50,000 cps. In some aspects, the crosslinked vinylpyrrolidone polymer can be prepared directly by precipitation polymerization of monomers consisting essentially of vinyl pyrrolidone in an organic solvent in the presence of a crosslinking agent (e.g., one or more of N,N′-divinylimidazolidone, pentaerythritol triallyl ether, triallyl-1,3,5-triazine-2,4,6(1H.3H,5H)-trione and 2,4,6-triallyloxy-1,3,5-triazine, or any other suitable crosslinking agent) in the amount of about 0.2 to about 1% by weight of vinyl pyrrolidone and a free radical polymerization initiator. In some aspects, the moderately crosslinked vinylpyrrolidone polymer can be prepared directly by precipitation polymerization of monomers consisting essentially of vinyl pyrrolidone in an organic solvent in the presence of a crosslinking agent in an amount of about 0.25-0.8%, or about 0.35-0.6% by weight of vinyl pyrrolidone and a free radical polymerization initiator (e.g., acyl peroxides such as diacetyl peroxide, dibenzoyl peroxide and dilauryl peroxide; peresters such as t-butylperoxy pivalate, tert-butyl peroxy-2-ethylhexanoate; peroxides such as di-tert-butyl peroxide; percarbonates such as dicyclohexyl peroxydicarbonate; and azo compounds such as 2.2′-azobis(isobutyronitrile), 2,2′-azobis(2.4-dimethylvaleronitrile), 1,1′-azobis(cyanocyclohexane), and 2,2′-azobis(methylbutyronitrile), or any other suitable initiator.

In some aspects, the strongly swellable, lightly to moderately crosslinked vinylpyrrolidone polymer can be made directly as fine, white powders comprising or consisting essentially of precipitation polymerizing vinyl pyrrolidone in the presence of a crosslinking agent and a free radical polymerization initiator in an organic solvent (e.g., an aliphatic hydrocarbon which is a C₃-C₁₀ saturated hydrocarbon, branched or unbranched, cyclic or acyclic, or mixtures thereof, or any other suitable organic solvent) at a polymerization temperature of about 40° C. to 150° C., or between about 60° C. to 70° C. In some aspects, the crosslinking agent can be present in an amount of about 0.2 to 1%, between 0.25-0.8%, or between 0.35-0.6% by weight of the vinyl pyrrolidone monomer. Pressure can be kept at atmospheric pressure, although higher and lower pressures may be used as well.

In some aspects, the at least one thickening agent comprises a substantially crosslinked, slightly swellable vinylpyrrolidone homopolymer which has similar chemical structure and hydrolytic degradation resistance as the strongly swellable, lightly to moderately crosslinked vinylpyrrolidone polymer. Examples include, but are not limited to Kollidon™ marketed by BASF, Mount Olive, N. J. USA, polyplasdone™ INF-10, marketed by Ashland Corporation, Wayne, N. J. USA.

In some aspects, the substantially crosslinked, slightly swellable vinylpyrrolidone polymer can be prepared directly by precipitation polymerization of monomers consisting essentially of vinyl pyrrolidone in an aqueous solution in the presence of a crosslinker but without any radical initiator. Suitable crosslinkers include 1-vinyl-3(E)-ethylidene pyrrolidone, ethylidene bisvinylpyrrolidone) and N,N′-divinylimidazolidone, triallyl-1,3,5-triazine-2,4,6 (1H, 3H, 5H)-trione, 2.4,6-triallyloxy-1,3,5-trizaine and pentaerthylthritol triallyl ether in an amount of about 0.5% to about 10% by weight of vinyl pyrrolidone, as described in U.S. Pat. Nos. 3,933,766 and 5,567,786.

In some aspects, the at least one thickening agent comprises a slightly crosslinked vinylpyrrolidone copolymer of an ammonium or alkali salt of acryloyldimethyltaurate and ethylene, acryloyldimethyltaurate and styrene or acryloyldimethyltaurate and vinylpyrrolidone, preferred are copolymers or mixtures of copolymers based on acryloyldimethyltaurate and at least one ethylenically unsaturated monomer selected from vinylpyrrolidone, styrene and vinylpolyethylene oxide. Available examples include but are not limited to Ultrathix P-100, and Lubrajel from Ashland Specialty (Wayne, NJ). Aristoflex AVC, Aristoflex AVS and Aristoflex HMB from Clariant International LTD. In some aspects, the at least one thickening agent is present in a range of about 0.1-20% by weight of the one-part tooth whitening composition. In some aspects, the at least one thickening agent is present in a range of about 2-12% by weight of the one-part tooth whitening composition. In some aspects, the at least one thickening agent is present in a range of about 4-10% by weight of the one-part tooth whitening composition. In some aspects, the at least one thickening agent is present in a range of about 4-9% by weight of the one-part tooth whitening composition.

### Liquid Carrier

The liquid carrier(s) or solvent(s) can comprise one or more of water, glycerin, propylene glycol, polyethylene glycol, sorbitol, ethanol and other suitable liquid carriers. In some aspects, the liquid carrier can comprise water, and the water can be present in an amount, for example, between 15-30 wt %, 15-50 wt %, or 30-95 wt % of the one-part composition. In some aspects, the liquid carrier can comprise at least one hygroscopic agent selected from the polyol group consisting of glycerin, propylene glycol, polyethylene glycol, polypropylene glycol, and sorbitol. The hygroscopic agent can be present in an amount, for example, between 1-85 wt %, between 30-70 wt %, or between 35-55 wt % of the one-part composition. The liquid carrier can be present in an amount, for example, between 30-95 wt %, between 30-85 wt %, between 50-85 wt %, or between 50-80 wt % of the one-part composition. In some embodiments, the liquid carriers comprise water and at least one hygroscopic agent, wherein the water can be present in an amount between, for example, 15-25 wt % of the one-part composition, and wherein the at least one hygroscopic agent is present in an amount between, for example, 35-55 wt %. In some aspects, the solvent is selected and present in an amount sufficient to form a gel whitening composition with suitable viscosity for teeth whitening purposes.

### Basic Agent

To avoid tooth etching and improve bleaching efficiency, it is preferable to add a pH adjuster, or more specifically, a base in order to increase the pH of tooth bleaching compositions, for example, to a pH range of about 5 to 9, a pH range of about 5-7, a pH range of about 7-9, or in some preferred aspects, a pH range of about 6 to 7, or 6.2 to 7.5. Compositions, kits, and systems of the disclosure can comprise any suitable pH adjuster, including, for example, a triethanolamine, sodium hydroxide, potassium hydroxide, ammonium hydroxide, or a combination thereof. In some embodiments, an aqueous potassium hydroxide can be a preferred pH adjuster. The pH adjuster may be present in any suitable amount in a composition, kit, or system. For example, the pH adjusters can be included in a range from about 1.0% to about 10.0% by weight of the one-part composition (or activated composition or part thereof), or in a range from about 2% to about 8% by weight of the one-part composition.

### Other Components

In some embodiments of the disclosed one-part compositions, kits, and systems, it may be desirable to include other components such as stabilizers, desensitizing agents, flavoring agents, sweeteners, anticariogenic agents, and antimicrobial agents.

The one-part tooth bleaching compositions, kits, and systems can include a poly(ethylene oxide)-poly(propylene oxide) block copolymer or a mixture of these copolymers to adjust the thixotropic properties of the composition, activated composition, or parts of a kit. In some aspects, a composition, kit or system of the disclosure can include a mixture of poly(ethylene oxide)-poly(propylene oxide)-poly(ethylene oxide) with a molecular weight range from 5000 g/mol to 15000 g/mol. An example includes a mixture of Poloxamer 407 and Poloxamer 188, also known as Pluronic F127 and Pluronic F68, respectively, which can be present in an amount of, for example, about 3-10 wt % of the composition (or activated composition or part thereof).

The one-part tooth bleaching compositions, kits, and systems can include a bleaching agent stabilizer, including, for example, ion scavengers, such as ethylenediaminetetraacetic acid disodium salt, ethylenediaminetetraacetic acid tetrasodium salt, ethylenediaminetetraacetic acid calcium disodium salt, citric acid, etidronic acid, succinic acid, sodium stannate, potassium stannate, sodium phosphate, sodium pyrophosphate, sodium acid pyrophosphate, or a combination or mixture thereof. In some aspects, the bleaching agent stabilizers can be included in an amount in a range from about 0% to about 1% by weight of the whitening composition, activated composition, or part(s) thereof, or from about 0.01% to about 0.5% by weight of the whitening composition, activated composition, or part(s) thereof.

The one-part tooth whitening compositions, kits, and systems can include one or more desensitizers (e.g., potassium nitrate (KNO3), sodium fluoride) to alleviate or prevent tooth sensitivity of patients or other users. In some aspects, the one or more desensitizers may be present in the composition in an amount from about 0.1-3%, or from about 0.3-0.8% by weight of the composition, the activated composition, or part(s) thereof.

The one-part tooth whitening compositions, kits, and systems can include one or more flavorants or sweeteners (e.g., peppermint, spearmint, corn mint, wintergreen, green tea, orange, lemon).

The one-part tooth whitening compositions, kits, and systems can include any additional ingredients suitable for human administration, for example, moisturizing agents, solubilizing agents, flow agents, preservatives, colors, flavors, and the like.

The one-part tooth whitening composition may be stored in syringes, pens, capsules or any suitable containers. The one-part tooth whitening composition can be applied to teeth by a brush, a swab, a sponge, or other suitable applicator.

### MULTI-PART TEETH WHITENING KITS AND SYSTEMS

In some aspects, a multi-part tooth whitening kit is provided, comprising a first part comprising a whitening agent, and a second part comprising an alkaline material, wherein at least one of the first and second parts comprises an effective amount of a crosslinked and/or water insoluble vinylpyrrolidone homo- or copolymer as a thickening agent. In some aspects, the first part comprises a peroxide as a whitening agent, the second part comprises a strong base dispersed in a stable gel, wherein the strong base comprises at least one of an alkali metal oxide, an alkali metal hydroxide, an alkaline earth metal oxide, and an alkaline earth metal hydroxide. The first part can have an acidic pH and include at least about 25 wt %, at least 30 wt %, at least 35 wt %, at least 40 wt %, or at least 45 wt % available hydrogen peroxide (or other suitable whitening agent). The second part can include at least one strong base dispersed in a stable gel comprising at least one polymeric thickening agent (e.g., a crosslinked and/or water insoluble vinylpyrrolidone polymer and/or vinylpyrrolidone containing copolymer). An apparatus for applying the composition, and instructions for determining a composition application time to achieve a whitening effect may be provided. In some aspects, the first and second parts can be placed in separate chambers of a dual-barrel syringe or other dispenser.

Viewed from another perspective, a multi-part bleaching system is provided that comprises a first whitening agent component or formulation and a second strong base component or formulation. In some aspects, a user (or in some embodiments, a container comprising a mixing tip), whether at home or in a dental office, can mix the two components in a desired ratio prior to use. The desired ratios can comprise, for example, from about 1:5 to about 10:1 (whitening agent component: strong base component), from about 1:1 to about 10:1 (whitening agent component: strong base component), from about 1:5 to about 5:1 (whitening agent component: strong base component), from about 1:1 to about 5:1 (whitening agent component: strong base component), from about 1:1 to about 5:1 (strong base component: whitening agent component), or any other suitable ratio.

### Whitening Agent Component (Peroxide Part; First Part)

The first part of the multi-part tooth whitening kit or system can include a dental bleaching agent and a polymeric thickener. In some aspects, a preferred whitening agent is hydrogen peroxide. Other whitening agents, such as carbamide peroxide, perborates are also contemplated. In order to generate strong bleach strength which is generally required for in-office whitening, high concentrations of aqueous hydrogen peroxide can be present in the composition. The amount of aqueous hydrogen peroxide (e.g. 50 wt % solution of hydrogen peroxide, 30-50 wt % solution of hydrogen peroxide), may be present in a range from about 50% to about 99% by weight of the composition in the peroxide part, preferably in a range from about 60% to about 96%, or from about 80-96 wt % by weight of composition in the peroxide part. In some aspects, the whitening agent (e.g., H₂O₂) can be present in a range of about 25-50%, or about 30-48%, or about 40-48% by weight of the composition in the peroxide part.

The peroxide part of the multi-part tooth whitening kit or system can also include at least one thickener in an amount from about 0.1 to about 20%, from about 0.1 to about 10%, from about 0.1 to about 8%, from about 5 to about 10%, from about 0.1 to about 15%, from about 1% to about 15%, or from about 6% to about 12% by weight of the composition. All suitable thickening agents are contemplated, preferably, a crosslinked and/or water insoluble vinylpyrrolidone homopolymer, or a crosslinked vinylpyrrolidone copolymer, such as FlexiThix™, Kollidon®, Aristoflex® AVC.

Other agents such as liquid carriers, fumed silica, stabilizers, dyes, and flavorants as described above in connection with the one-part compositions may also be included as desired. Contemplated peroxide parts can comprise a desensitizing agent, for example, a potassium nitrate, a sodium fluoride, or a mixture thereof. Contemplated peroxide parts can comprise one or more bleaching agent stabilizers, for example, ethylenediaminetetraacetic acid disodium salt. ethylenediaminetetraacetic acid tetrasodium salt, ethylenediaminetetraacetic acid calcium disodium salt, citric acid, ctidronic acid, succinic acid, sodium stannate, potassium stannate, sodium phosphate, sodium pyrophosphate, sodium acid pyrophosphate, or a mixture thereof. In some aspects, the bleaching agent stabilizer can be present in an amount of between 0.1-2 wt %, between 0.1-1 wt %, or between 0.01-0.5 wt %, no more than 2 wt %, no more than 1 wt %, or no more than 0.5 wt % of the first part and/or combination of first and second parts. Contemplated peroxide parts can comprise a poly(ethylene oxide)-poly(propylene oxide) block copolymer that is present in an amount of between 1 wt % and 10 wt % of the first part and/or combination of first and second parts.

In some aspects, the peroxide part can have a pH below 7, more preferably below 6, below 5, below 4, below 3, between 1 and 4, or between 1 and 3 for stability of the whitening agent.

### Strong Base Component (Activation Part; Second Part)

The second part of the multi-part tooth whitening kit or system can comprise a strong base gel which can include a liquid carrier, a strong base, and a polymeric thickener. It may also include a desensitizer, a flavorant, or a dye, as desired. The strong base gel can be used to increase the pH of the tooth whitening kit or system to a desired value and activate whitening when mix with the peroxide part.

The one or more liquid carriers can comprise water, ethanol and/or liquid polyols such as propylene glycol and polyethylene glycol, or any other suitable carrier(s). The liquid carrier(s) can be present in an amount, for example, from about 10-99 wt %, from about 50-95 wt %, or from about 60-90 wt % of the activation part. In some aspects, the one or more liquid carriers comprise water and polyethylene glycol, wherein water can be present in an amount, for example, from about 10% to about 95% by weight, from about 20% to about 90% by weight, from about 30% to about 70% by weight, from about 40% to about 60% by weight, from about 10% to about 50% by weight, from about 10% to about 40% by weight, from about 30% to about 50% by weight of the activation part, and wherein polyethylene glycol can be present in an amount, for example, from about 10% to about 60% by weight, preferably in a range from about 40% to about 60%, or from about 35% to about 50% by weight of the activation part. The polyethylene glycol can have a molecular weight of, for example, about 200 to about 600 g/mol. In some aspects, the one or more liquid carriers comprise water, and the water is present in an amount, for example, from about 10% to about 95% by weight, from about 20% to about 90% by weight, from about 30% to about 70% by weight, from about 40% to about 60% by weight, from about 10% to about 50% by weight, from about 10% to about 40% by weight, from about 30% to about 50% by weight of the activation part.

Strong bases suitable for this invention include hydroxides and carbonates of alkali which dissolve in the liquid carrier. Examples of strong bases includes sodium hydroxide and potassium hydroxide. In some aspects, the strong bases can be present in the activation part in any suitable amount, including for example, an amount in a range from about 1% to about 4% by the weight, or in a range from about 1.5% to about 4% by weight of the activation part.

The activation part of the multi-part tooth whitening kit or system can also include at least one thickener, for example, in an amount from about 0.1 to about 20% by weight, from about 0.1 to about 10% by weight. 0.1 to about 8% by weight, 0.1 to about 15% by weight, 1% to about 15% by weight of the activation part, and in some embodiments in a range from about 6% to about 12%, from about 3% to about 8%, or from about 4% to about 6% by weight of the activation part. All suitable thickening agents are contemplated, including, for example, a crosslinked and/or water insoluble vinylpyrrolidone homo- or copolymer. In some aspects, a crosslinked vinylpyrrolidone homopolymer, for example, a lightly to moderately crosslinked PVP, which is capable of thickening in broad pH ranges, is preferred.

Contemplated activation parts can also comprise a desensitizing agent, for example, a potassium nitrate, a sodium fluoride, or a mixture thereof. Contemplated activation parts can comprise a poly(ethylene oxide)-poly(propylene oxide) block copolymer that is present in an amount of between 1 wt % and 10 wt %, or between 4 wt % and 8 wt % of the activation part. Contemplated activation parts can comprise one or more pH adjusting agents, for example, triethanolamine, sodium hydroxide, potassium hydroxide, or ammonium hydroxide. The one or more pH adjusting agents can be present in an amount of, for example, 0.01-10 wt % or 0.01-5 wt % of the activation part. Some activation part can have a pH of, for example, at least 10, or at least 12, or at least 14. Contemplated activation parts can also comprise any other agents such as stabilizers, dyes, flavorants, and/or other agents as described above.

The peroxide part or the activation part can include any additional ingredients suitable for human administration, for example, moisturizing agents, solubilizing agents, flow agents, preservatives, colors, flavors, and the like. In some aspects, the peroxide part and the activation part can include different colorants (e.g., dyes, pigments, lakes) such that the parts have different colors from one another for easy identification.

### Mixed Composition from Peroxide Part and Activation Part (Activated Composition)

The peroxide part and activation part can be stored separately. For example, a tooth whitening kit that includes a peroxide part and an activation part in separate containers, or a dual barrel syringe that stores a peroxide part and an activation part separately. The peroxide part and activation part may be mixed in any suitable mode prior to use. For example, auto mixed with a dual barrel mixing tip or hand mixed two parts from separate containers.

The peroxide part and activation part may be mixed in any desired ratios for example, from about 1:5 to about 10:1 (peroxide part: activation part), from about 1:1 to about 10:1 (peroxide part: activation part), from about 1:5 to about 5:1 (peroxide part: activation part), from about 1:1 to about 5:1 (peroxide part: activation part), from about 1:1 to about 5:1 (activation part: peroxide part), from about 3.5:1 to about 4.5:1 (peroxide part: activation part), about 4:1 (peroxide part: activation part), or any other suitable ratio, to form an activated composition. The pH of the mixed composition, which may be a gel, can be in the range of 5 to 9, and in some preferred aspects, a range of 7 to 9. Once the parts are mixed, the mixed tooth bleaching composition may be applied to at least one tooth using any known application method or applicator. The kit can comprise a brush, a swab, a sponge, or other suitable applicator for applying or spreading the combined parts onto a user's teeth. The mixed whitening composition may remain on the tooth for any suitable period of time, for example, a period of time ranging from about 1 minute to about 30 minutes, from about 5 minutes to about 25 minutes, or from about 8 minutes to about 20 minutes.

In some embodiments, the activated composition can comprise one or more liquid carriers, such as deionized water and/or polyethylene glycol. In some embodiments, the one or more liquid carriers can be present in the activated composition in any suitable amount (e.g., between 20-75%, between 30-75%, between 40-75%, between 45-65%, at least 35%, at least 40%, at least 45%, at least 50% by weight of the activated composition). In some embodiments, the one or more liquid carriers can comprise water, and the water can be present in the activated composition in any suitable amount (e.g., between 20-75%, between 30-75%, between 35-55%, at least 20%, at least 25%, at least 30%, at least 35%, at least 40% by weight of the activated composition). In some embodiments, the one or more liquid carriers can comprise polyethylene glycol, and the polyethylene glycol can be present in the activated composition in any suitable amount (e.g., between 1-25%, between 1-20%, between 5-15% by weight of the activated composition). In some embodiments, the one or more liquid carriers can comprise water, glycerin, propylene glycol, polyethylene glycol, sorbitol, ethanol and/or other suitable liquid carriers in any suitable amount. In some embodiment, the activated composition can comprise one or more whitening components, and the one or more whitening components can be present in the activated composition in any suitable amount (e.g., hydrogen peroxide and/or other whitening component present in an amount of between 20-60%, between 25-55%, between 30-45%, at least 20%, at least 30%, at least 35% by weight of the activated composition). In some embodiment, the activated composition can comprise one or more thickening agents, and the one or more thickening agents can be present in the activated composition in any suitable amount (e.g., crosslinked (e.g., lightly to moderately, highly) PVP and/or other thickening agents present in an amount of between 0.1-15%, between 1-15%, between 5-10% by weight of the activated composition). In some embodiments, the activated composition can comprise a pH adjuster (e.g., sodium hydroxide and/or other suitable pH adjusters) such that the pH of the activated composition is, for example, at least 7. In some embodiments, the activated composition can include any additional ingredients suitable for human administration, for example, desensitizing agents (e.g., potassium nitrate in an amount of between 0.1-5%, 0.1-1.5% by weight of the activated composition), moisturizing agents, solubilizing agents, flow agents, preservatives, colors, flavors, and/or any other suitable ingredients.

### EXAMPLES

The examples below are presented for illustrative purposes. Exemplary one-part tooth whitening compositions are displayed in Table 1. The tooth whitening compositions disclosed herein may, in certain embodiments, be prepared by adding and mixing the ingredients of the composition in a suitable vessel, such as a stainless steel tank with a mixer. In the preparation, the ingredients may be added to the mixer in the following sequence: salts, deionized water, polyols, peroxide compounds, powdered thickening agents, pH adjusting agent and any desired flavoring agents and/or sweeteners. The ingredients were mixed to form a homogeneous gel. Each of the tooth whitening compositions was deaerated and then packed in 3 cc clear LDPE Luer-lock syringes for stability evaluation.

The finished compositions were tested for hydrogen peroxide content, viscosity, and pH.

Hydrogen peroxide determination: Hydrogen peroxide concentration, expressed as weight percentage of the total gel composition weight, was determined by a standard iodometric titration (Solvay technical data sheet, determination of hydrogen peroxide concentration).

Viscosity determination: The viscosity of the whitening compositions were determined by using a Brookfield R/S Plus viscometer with a cone-plate attachment. The spindle RCT-50-1 was used at spindle speed of 100 rpm at 25° C.

pH determination: The pH was measured by a pH meter of the Accumet Model AB250 (Fisher Scientific) with a flat pH probe (Thermo Scientific™ 9135APWP). The pH meter was calibrated with standard pH buffer solutions at pH 4 and pH 7 before measurement. The pH value of the whitening composition was obtained by inserting the pH probe into the whitening gel and reading the pH value when it was stabilized.

Table 1 sets forth 6 Examples of one-part tooth whitening gel compositions of the disclosure.

TABLE 1
Compositions of Representative Tooth Whitening Gels

| | Example 1 | Example 2 | Example 3 | Example 4 | Example 5 | Example 6 |
|---|---|---|---|---|---|---|
| Ingredient | wt % | wt % | wt % | wt % | wt % | wt % |
| Water | 19.6 | 19.6 | 25.1 | 21.2 | 25.0 | 20.0 |
| Polyethylene | 45.4 | 40.5 | 49.3 | 50.0 | 39.8 | 38.8 |
| glycol | | | | | | |
| Propylene | — | — | — | — | — | 15.0 |
| glycol | | | | | | |
| Hydrogen | 19.6 | 19.6 | 16.7 | 19.8 | 28.0 | 18.0 |
| peroxide | | | | | | |
| (50%) | | | | | | |
| FlexiThix | 8.8 | 8.8 | 7.5 | — | 5.0 | 6.0 |
| Aristoflex | — | — | — | 2.0 | — | — |
| Poloxamer | 4.9 | 8.8 | — | 5 | — | — |
| Disodium | 0.2 | 0.20 | 0.2 | 0.2 | 0.2 | 0.2 |
| EDTA | | | | | | |
| KNO₃ | 0.5 | 0.5 | 0.4 | — | 0.5 | 0.5 |
| Potassium | To pH | To pH | To pH | To pH | to pH | to pH |
| hydroxide | 5.6 | 6.3 | 5.4 | 5.8 | 6.3 | 7.0 |
| (0.5N) | | | | | | |
| Flavor | >0.05 | >0.05 | >0.05 | >0.05 | >0.05 | >0.05 |
| Total | 100 | 100 | 100 | 100 | 100 | 100 |

Each of the compositions above were viscous but not stringy. They could be easily dispensed from a syringe. The compositions exhibited high yield stress which can prevent the compositions running from the treated tooth surface to the surrounding soft tissue.

An example of a two-part tooth whitening system which includes the components in Peroxide Part and Activation Part is displayed in Table 2, with Peroxide Part and Activation Part being two separate parts that are combined prior to use to form an activated whitening composition. The kit or system was designed to combine Peroxide Part and Activation Part in a volume ratio of about 4 to 1 (or between 3.5:1 and 4.5:1 Peroxide Part to Activation Part) to form an activated bleaching composition prior to use.

TABLE 2
Two-part tooth bleaching composition

| | Example 7 | | |
|---|---|---|---|
| | | | Mixture of |
| | | | Peroxide |
| | | | Part and |
| | Peroxide | Activation | Activation |
| | Part | part | Part in 4:1 |
| Ingredient | wt (%) | wt (%) | ratio (wt %) |
| Deionized water | — | 42.00 | 43.1 |
| Polyethylene glycol | — | 45.00 | 10.9 |
| H₂O₂ (50%) | 91.00 | — | — |
| H₂O₂ (100%) | — | — | 36.5 |
| Crosslinked PVP | 9.00 | 5.00 | 7.7 |
| (FlexiThix) | | | |
| NaOH | — | to | to |
| | | pH > 12 | pH > 7.0 |
| KNO₃ | — | 6.00 | 0.8 |
| Colorant | — | <0.10 | <0.10 |
| Total | 100.00 | 100.00 | 100.00 |

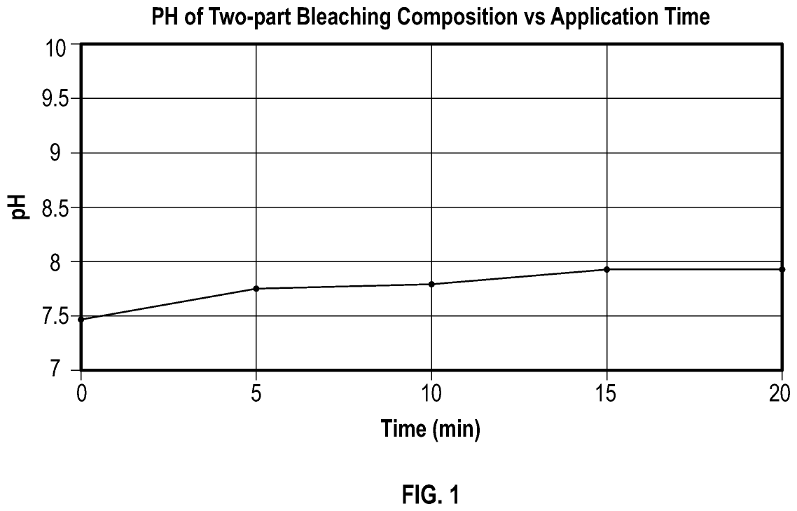

Each of Parts were stable viscous gels but not tacky. The Peroxide Part and Activation Part were mixed and formed an activated whitening composition that included the foregoing components in a volume ratio of 4 to 1. The pH of the immediately mixed composition was above 7.4 and was increased to 7.9 in 20 minutes after mixing, as shown in FIG. 1. The mixed composition remained viscous but not tacky during dispensing. It was easily dispensed on tooth.

### Stability of Hydrogen Peroxide at an Accelerated Aging

The aging stability of tooth bleaching composition is very important to the product's performance and efficacy. Accelerated aging testing is commonly used to justify shelf life of new products when room temperature stability tests would delay its introduction to market. Table 3 shows the properties of the tooth bleaching compositions of Examples 1-3, and 5-6 stored at 60° C., as well as Example 4 and 7 stored at 50° C. It shows a loss of less than 10% of the initial hydrogen peroxide content for Examples 1-7 after the aging period. There was no significant change in viscosity of Example 1, 5 and 6. Examples 2, 3, 4 and 7 lost 9%, 13%, 16% and 19% of its initial viscosity respectively after the aging period. With respect to Example 7, the two parts were stored separately during the aging period of 8 weeks at 50° C., and the retention rate of HP and retention rate of viscosity were tested immediately after mixing. In contrast, the commercial whitening 1 and 3 substantially lost its initial hydrogen peroxide content. Commercial whitening 1 and 2 lost all its initial viscosity after 4 weeks of aging at 60° C. Because oral care compositions surviving up to 13 weeks of aging at 40° C. is used to predict a 24 months shelf life under room temperature, likewise, 4 weeks of storage at 60° C. is reliable projected to 14 weeks of storage at 47° C., or 29 weeks at 40° C., or a minimum of three years storage at room temperature without refrigeration. These results indicate the examples1-7 are surprisingly stable in aging.

TABLE 3
Retention rate of hydrogen peroxide (HP)
and viscosity of whitening compositions

| | Aging | Aging | Retention | Retention |
|---|---|---|---|---|
| | temperature | period | rate of | rate of |
| Group | (° C.) | (weeks) | HP (%) | viscosity (%) |
| Example 1 | 60 | 8 | 96% | 102% |
| Example 2 | 60 | 8 | 91% | 91% |
| Example 3 | 60 | 6 | 92% | 87% |
| Example 4 | 50 | 10 | 92% | 84% |
| Example 5 | 60 | 4 | 93% | 101% |
| Example 6 | 60 | 4 | 97% | 104% |
| Example 7 | 50 | 8 | 97% | 81% |
| Commercial | 60 | 4 | 4% | 0% |
| whitening 1 | | | | |
| Commercial | 60 | 4 | 84% | 0% |
| whitening 2 | | | | |
| Commercial | 60 | 4 | 1% | 81% |
| whitening 3 | | | | |

Enamel like hydroxyapatite (HA) discs (ø=12 mm, Himed, Old Bethpage, NY) were stained using a tea-staining protocol. Before staining, the HA discs were polished by 600-grit sand papers and cleaned with distilled water. They were then immersed in a solution of black tea (Lipton black tea bags, USA) for a month at 37° C. with tea refreshing in every 2-3 days in order to create a stained surface. The tea solution was created by soaking one filter bag in eight ounces of boiling water for five minutes.

Each stained HA disc was measured for color data L, a, b value using a VITA Easyshade V (VITA North America). HA discs were then painted with the tooth whitening compositions listed in Table 4. All gels were kept in contact with the HA disc surface for 10 minutes in a humidor at 37° C. simulated an oral environment and then rinsed gel residue away with distilled water. This procedure was then repeated for three more times. The degree of whitening was thereafter determined by measuring the color change and recorded as JE. This color change is defined as the following equation:

$$Δ E = \sqrt{{( Δ L )}^{2} + {( Δ a )}^{2} + {( Δ b )}^{2}} \qquad ( 2 )$$

where L indicates lightness, a and b indicate the chromaticity coordinates.

TABLE 4
Color change of HA discs with commercial whitening products and an example composition

| | Total treatment | Initial color | | | Final color | | | |
|---|---|---|---|---|---|---|---|---|
| Product/example | time (minutes) | L | a | b | L | a | b | ΔE |
| Commercial whitening 1 | 40 | 62.3 | 6.0 | 21.2 | 70.4 | 3.3 | 20.1 | 14.8 |
| Commercial whitening 2 | 40 | 60.6 | 6.6 | 22 | 68.4 | 4.1 | 20.5 | 8.3 |
| Commercial whitening 3 | 40 | 62.0 | 6.4 | 24.5 | 71.9 | 0.8 | 15.0 | 8.6 |
| Example 2 | 40 | 63.1 | 5.9 | 20.8 | 76.1 | 1.0 | 16.3 | 14.6 |

As shown in Table 4, the ΔE of the HA disc treated with the Example 2 and Commercial whitening 1 were 14.6 and 14.8, respectively. In contrast, the ΔE of both Commercial whitening 2 and 3 were below 9. These results indicate the overall whitening efficacy of example 2 was similar as the commercial whitening 1 and better than commercial whitening 2 and 3.

### NON-LIMITING EMBODIMENTS

Embodiment 1. A ready to use, one part, aqueous tooth whitening composition, comprising at least one whitening agent present in an amount ranging from about 3-50 wt % of the composition, at least one thickening agent comprising at least one of a crosslinked and/or water insoluble N-vinyl heterocylic homopolymer and a crosslinked and/or water insoluble N-vinyl heterocyclic copolymer, the at least one thickening agent present in amount ranging from about 0.1%-15 wt % of the composition, a liquid carrier, wherein the liquid carrier comprises water present in an amount of at least 10 wt % of the composition, and wherein the composition is formulated to maintain at least 90% available hydrogen peroxide content and at least 80% viscosity after 4 weeks storage at 60° C.

Embodiment 2. The ready to use, one part, aqueous tooth whitening composition of embodiment 1, wherein the composition has a pH of at least about 6.0.

Embodiment 3. The ready to use, one part, aqueous tooth whitening composition of any of embodiments 1-2, wherein the composition has a pH of at least about 6.5.

Embodiment 4. The ready to use, one part, aqueous tooth whitening composition of any of embodiments 1-3, wherein the at least one thickening agent comprises a crosslinked vinylpyrrolidone polymer and/or vinylpyrrolidone containing copolymer.

Embodiment 5. The ready to use, one part, aqueous tooth whitening composition of any of embodiments 1-4, wherein the composition is formulated to maintain at least 90% available hydrogen peroxide content and at least 80% viscosity after 24 months of storage at 23° C.

Embodiment 6. The ready to use, one part, aqueous tooth whitening composition of any of embodiments 1-5, wherein the at least one whitening agent comprises at least one of hydrogen peroxide and urea hydrogen peroxide, and wherein the at least one whitening agent is present in an amount ranging from 6-25 wt %.

Embodiment 7. The ready to use, one part, aqueous tooth whitening composition of any of embodiments 1-6, wherein the at least one whitening agent comprises at least one of hydrogen peroxide and urea hydrogen peroxide, and wherein the at least one whitening agent is present in an amount ranging from 9-15 wt %.

Embodiment 8. The ready to use, one part, aqueous tooth whitening composition of any of embodiments 1-7, wherein the composition is formulated to maintain at least 90% available hydrogen peroxide content and at least 80% viscosity after 36 months of storage at 23° C.

Embodiment 9. The ready to use, one part, aqueous tooth whitening composition of any of embodiments 1-8, wherein the at least one thickening agent present in amount ranging from about 0.1%-10 wt % of the composition.

Embodiment 10. The ready to use, one part, aqueous tooth whitening composition of any of embodiments 1-9, wherein the liquid carrier comprises water present in an amount of at least 50 wt % of the composition.

Embodiment 11. The ready to use, one part, aqueous tooth whitening composition of any of embodiments 1-10, wherein the liquid carrier comprise at least one hygroscopic agent selected from the polyol group consisting of glycerin, propylene glycol, polyethylene glycol, polypropylene glycol and sorbitol, and wherein the hygroscopic agent comprises from 1.0 wt % to 85 wt % of the composition.

Embodiment 12. The ready to use, one part, aqueous tooth whitening composition of any of embodiments 1-11, wherein the composition is a gel.

Embodiment 13. A tooth whitening kit, comprising a first part comprising a whitening agent. a second part comprising an alkaline material, wherein at least one of the first and second parts comprises an effective amount of at least one thickening agent, wherein the at least one thickening agent comprises at least one of a crosslinked N-vinyl heterocyclic homopolymer and a crosslinked N-vinyl heterocyclic copolymer, and wherein combining the first part and the second part forms an activated tooth whitening composition with a pH of between 7 and 9.

Embodiment 14. The tooth whitening kit of embodiment 13, wherein the whitening agent comprises a peroxide compound, and wherein the second part comprises a strong base dispersed in a stable gel, wherein the strong base comprises at least one of an alkali metal oxide, an alkali metal hydroxide, an alkaline earth metal oxide, and an alkaline earth metal hydroxide.

Embodiment 15. The tooth whitening kit of any of embodiments 13-14, wherein the activated tooth whitening composition is formed by combining the first part and the second part in a volume ratio of between 3.5:1 and 4.5:1.

Embodiment 16. The tooth whitening kit of any of embodiments 13-15, wherein the peroxide compound is hydrogen peroxide and the concentration of peroxide compound present in the combined whitening component ranges from 25 wt % to about 40 wt %.

Embodiment 17. The tooth whitening kit of any of embodiments 13-16, wherein the strong base is selected from the group consisting of triethanolamine, sodium hydroxide, potassium hydroxide, and ammonium hydroxide.

Embodiment 18. The tooth whitening kit of any of embodiments 13-17, wherein a pH of the activated tooth whitening composition increases during the first twenty minutes after the activated tooth whitening composition is formed.

Embodiment 19. The tooth whitening kit of any of embodiments 13-18, wherein each of the first and second parts comprises the at least one thickening agent.

Embodiment 20. The tooth whitening kit of any of embodiments 13-19, further comprising a dual barrel syringe that stores the first part and second part separately.

Embodiment 21. The tooth whitening kit of any of embodiments 13-20, wherein the dual barrel syringe comprises a mixing tip.

Embodiment 22. The tooth whitening kit of any of embodiments 13-21, wherein the second part comprises one or more liquid carriers, and wherein the one or more liquid carriers are present in an amount of about 50-90 wt % of the second part.

Embodiment 23. The tooth whitening kit of any of embodiments 13-22, wherein the second part has a pH of at least 12.

Embodiment 24. The tooth whitening kit of any of embodiments 13-23, wherein the at least one thickening agent is present in the first part in an amount from about 5 to about 10 wt %.

Embodiment 25. The tooth whitening kit of any of embodiments 13-24, wherein the kit is an at-home or in-office kit.

Embodiment 26. The tooth whitening kit of any of embodiments 13-25, wherein each of the first and second parts are formulated such that when stored separately for a period of 8 weeks at 50° C., then combined, the activated tooth whitening composition comprises at least 90% available hydrogen peroxide content and at least 80% viscosity relative to the available hydrogen peroxide and viscosity if combined prior to storage for the at least 8 weeks storage at 50° C.

Embodiment 27. A ready to use, one part, aqueous tooth whitening composition, comprising at least one whitening agent present in an amount ranging from about 3-50 wt % of the composition, at least one thickening agent comprising at least one of a water insoluble N-vinyl heterocylic homopolymer and a water insoluble N-vinyl heterocyclic copolymer, the at least one thickening agent present in amount ranging from about 0.1-15 wt % of the composition, a liquid carrier, wherein the liquid carrier comprises water present in an amount of at least 10 wt % of the composition, and wherein the composition is formulated to maintain at least 90% available hydrogen peroxide content and at least 80% viscosity after 4 weeks storage at 60° C.

Embodiment 28. The ready to use, one part, aqueous tooth whitening composition of embodiment 27, wherein the composition has a pH of at least about 6.0.

Embodiment 29. The ready to use, one part, aqueous tooth whitening composition of any of embodiments 27-28, wherein the composition has a pH of at least about 6.5.

Embodiment 30. The ready to use, one part, aqueous tooth whitening composition of any of embodiments 27-29, wherein the at least one thickening agent comprises a crosslinked vinylpyrrolidone polymer and/or vinylpyrrolidone containing copolymer.

Embodiment 31. The ready to use, one part, aqueous tooth whitening composition of any of embodiments 27-30, wherein the composition is formulated to maintain at least 90% available hydrogen peroxide content and at least 80% viscosity after 24 months of storage at 23° C.

Embodiment 32. The ready to use, one part, aqueous tooth whitening composition of any of embodiments 27-31, wherein the at least one whitening agent comprises at least one of hydrogen peroxide and urea hydrogen peroxide, and wherein the at least one whitening agent is present in an amount ranging from 6-25 wt %.

Embodiment 33. The ready to use, one part, aqueous tooth whitening composition of any of embodiments 27-32, wherein the at least one whitening agent comprises at least one of hydrogen peroxide and urea hydrogen peroxide, and wherein the at least one whitening agent is present in an amount ranging from 9-15 wt %.

Embodiment 34. The ready to use, one part, aqueous tooth whitening composition of any of embodiments 27-33, wherein the composition is formulated to maintain at least 90% available hydrogen peroxide content and at least 80% viscosity after 36 months of storage at 23° C.

Embodiment 35. The ready to use, one part, aqueous tooth whitening composition of any of embodiments 27-34, wherein the at least one thickening agent present in amount ranging from about 0.1%-10 wt % of the composition.

Embodiment 36. The ready to use, one part, aqueous tooth whitening composition of any of embodiments 27-35, wherein the liquid carrier comprises water present in an amount of at least 50 wt % of the composition.

Embodiment 37. The ready to use, one part, aqueous tooth whitening composition of any of embodiments 27-36, wherein the liquid carrier comprise at least one hygroscopic agent selected from the polyol group consisting of glycerin, propylene glycol, polyethylene glycol, polypropylene glycol and sorbitol, and wherein the hygroscopic agent comprises from 1.0 wt % to 85 wt % of the composition.

Embodiment 38. The ready to use, one part, aqueous tooth whitening composition of any of embodiments 27-37, wherein the composition is a gel.

Thus, specific examples of teeth whitening compositions, kits, and systems have been disclosed. It should be apparent, however, to those skilled in the art that many more modifications besides those already described are possible without departing from the inventive concepts herein. While examples and variations of the many aspects of the invention have been disclosed and described herein, such disclosure is provided for purposes of explanation and illustration only. Thus, various changes and modifications may be made without departing from the scope of the claims.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A ready to use, one part, aqueous tooth whitening composition, comprising:
at least one whitening agent present in an amount ranging from about 3-50 wt % of the composition;
at least one thickening agent comprising at least one of a crosslinked N-vinyl heterocylic homopolymer and a crosslinked N-vinyl heterocyclic copolymer, the at least one thickening agent present in amount ranging from about 0.1%-15 wt % of the composition;
a liquid carrier, wherein the liquid carrier comprises water present in an amount of at least 10 wt % of the composition; and
wherein the composition is formulated to maintain at least 90% available hydrogen peroxide content and at least 80% viscosity after 4 weeks storage at 60° C.

2. The composition of claim 1, wherein the composition has a pH of at least about 6.0.

3. The composition of claim 1, wherein the composition has a pH of at least about 6.5.

4. The composition of claim 1, wherein the at least one thickening agent comprises a crosslinked vinylpyrrolidone polymer and/or vinylpyrrolidone containing copolymer.

5. The composition of claim 1, wherein the composition is formulated to maintain at least 90% available hydrogen peroxide content and at least 80% viscosity after 24 months of storage at 23° C.

6. The composition of claim 1, wherein the at least one whitening agent comprises at least one of hydrogen peroxide and urea hydrogen peroxide, and wherein the at least one whitening agent is present in an amount ranging from 6-25 wt %.

7. The composition of claim 1, wherein the at least one whitening agent comprises at least one of hydrogen peroxide and urea hydrogen peroxide, and wherein the at least one whitening agent is present in an amount ranging from 9-15 wt %.

8. The composition of claim 1, wherein the composition is formulated to maintain at least 90% available hydrogen peroxide content and at least 80% viscosity after 36 months of storage at 23° C.

9. The composition of claim 1, wherein the at least one thickening agent present in amount ranging from about 0.1-10 wt % of the composition.

10. The composition of claim 1, wherein the liquid carrier comprises water present in an amount of at least 50 wt % of the composition.

11. The composition of claim 1, wherein the liquid carrier comprise at least one hygroscopic agent selected from the polyol group consisting of glycerin, propylene glycol, polyethylene glycol, polypropylene glycol and sorbitol, and wherein the hygroscopic agent comprises from 1.0 wt % to 85 wt % of the composition.

12. The composition of claim 1, wherein the composition is a gel.

13. A tooth whitening kit, comprising:
a first part comprising a whitening agent; and
a second part comprising an alkaline material;
wherein at least one of the first and second parts comprises an effective amount of at least one thickening agent, wherein the at least one thickening agent comprises at least one of a crosslinked N-vinyl heterocyclic homopolymer and a crosslinked N-vinyl heterocyclic copolymer, and wherein combining the first part and the second part forms an activated tooth whitening composition with a pH of between 7 and 9.

14. The tooth whitening kit of claim 13, wherein the whitening agent comprises a peroxide compound, and wherein the second part comprises a strong base dispersed in a stable gel, wherein the strong base comprises at least one of an alkali metal oxide, an alkali metal hydroxide, an alkaline earth metal oxide, and an alkaline earth metal hydroxide.

15. The tooth whitening kit of claim 13, wherein the activated tooth whitening composition is formed by combining the first part and the second part in a volume ratio of between 3.5:1 and 4.5:1.

16. The tooth whitening kit of claim 13, wherein the peroxide compound is hydrogen peroxide and the concentration of peroxide compound present in the combined (activated) whitening component ranges from 25 wt % to about 40 wt %.

17. The tooth whitening kit of claim 13, wherein the strong base is selected from the group consisting of triethanolamine, sodium hydroxide, potassium hydroxide, and ammonium hydroxide.

18. The tooth whitening kit of claim 13, wherein a pH of the activated tooth whitening composition increases during the first twenty minutes after the activated tooth whitening composition is formed.

19. The tooth whitening kit of claim 13, wherein each of the first and second parts comprises the at least one thickening agent.

20. The tooth whitening kit of claim 13, further comprising a dual barrel syringe that stores the first part and second part separately.

21. The tooth whitening kit of claim 20, wherein the dual barrel syringe comprises a mixing tip.

22. The tooth whitening kit of claim 13, wherein the second part comprises one or more liquid carriers, and wherein the one or more liquid carriers are present in an amount of about 50-90 wt % of the second part.

23. The tooth whitening kit of claim 13, wherein the second part has a pH of at least 12.

24. The tooth whitening kit of claim 13, wherein the at least one thickening agent is present in the first part in an amount from about 5 to about 10 wt %.

25. The tooth whitening kit of claim 13, wherein the kit is an at-home or in-office kit.

26. The tooth whitening kit of claim 13, wherein each of the first and second parts are formulated such that when stored separately for a period of 8 weeks at 50° C., then combined, the activated tooth whitening composition comprises at least 90% available hydrogen peroxide content and at least 80% viscosity relative to the available hydrogen peroxide and viscosity if combined prior to storage for the at least 8 weeks storage at 50° C.

27. A ready to use, one part, aqueous tooth whitening composition, comprising:
at least one whitening agent present in an amount ranging from about 3-50 wt % of the composition;
at least one thickening agent comprising at least one of a water insoluble N-vinyl heterocyclic homopolymer and a water insoluble N-vinyl heterocyclic copolymer, the at least one thickening agent present in amount ranging from about 0.1-15 wt % of the composition;
a liquid carrier, wherein the liquid carrier comprises water present in an amount of at least 10 wt % of the composition; and
wherein the composition is formulated to maintain at least 90% available hydrogen peroxide content and at least 80% viscosity after 4 weeks storage at 60° C.
